(19) United States
(12) Patent Application Publication
(10) Pub. No.: US 2003/0126692 A1
(43) Pub. Date: Jul. 10, 2003

(54) Use of particular aminosilicones as a pre-treatment of processes for coloring keratin fibers with direct dyes or with oxidation dyes

(76) Inventors: Priscille Devin-Baudoin, Vanves (FR); Anne Sabbagh, Rueil Malmaison (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,149

(22) Filed: Nov. 8, 2002

(30) Foreign Application Priority Data
Nov. 8, 2001 (FR) 01 14484

(51) Int. Cl. A61K 7/13

(52) U.S. Cl. 8/405; 8/406; 8/407

(57) ABSTRACT

A pre-treatment of a process for coloring, such as with at least one coloring agent chosen from direct dyes and oxidation dyes, human keratin fibers such as human hair, of a composition comprising at least one particular aminosilicone as well as the processes for coloring, with, for example, at least one coloring agent chosen from direct dyes and oxidation dyes, human keratin fibers such as hair, comprising a pre-treatment with a composition comprising at least one particular aminosilicone. The compound itself for pre-treatment.

This disclosure relates to the use, as a pre-treatment for a process for coloring human keratin fibers such as hair with at least one coloring agent chosen from oxidation dyes and direct dyes, of a composition comprising at least one particular aminosilicone. The disclosure also relates to a composition itself for pre-treatment.

This disclosure also relates to a process for coloring, with at least one coloring agent chosen from oxidation dyes and direct dyes, human keratin fibers such as hair, comprising a pretreatment with a composition comprising at least one particular aminosilicone.

Two main types of processes for coloring keratin fibers exist: direct dyeing, using, in the presence or absence of oxidizing agents, direct dyes and/or pigments which are colored molecules, giving the fibers a temporary color that may fade out after shampooing a few times, and “oxidation dyeing” using oxidation dye precursors and an oxidizing agent, which may give the fibers a more resistant color than that obtained with the previous type of dyeing.

There is a need to improve the rise of these colorations on fibers, for example, sensitized fibers, since they are more porous and they fix the colorants less.

Moreover, the use of an oxidizing agent generally may result in a certain level of degradation of the keratin fiber.

There is thus a need to limit these degradations and the consequences they may entail on the cosmetic condition of the fiber.

After extensive research, the inventors have discovered, entirely surprisingly and unexpectedly, that the use, as a pre-treatment on human keratin fibers such as hair, of a composition comprising at least one particular aminosilicone, may allow this problem to be solved. This discovery forms at least a portion of the basis for at least one embodiment disclosed herein.

In addition, this pre-treatment may improve the resistance of colorations with direct dyes or with oxidation dyes, for example, with respect to shampooing.

One new embodiment therefore relates to the use, as a pre-treatment of a process for coloring human keratin fibers such as hair with at least one coloring agent chosen from oxidation dyes and direct dyes, of a composition comprising at least one at least one amino silicone comprising at least one aminoethylimino(C₄-C₈)alkyl group.

In one embodiment, said composition may improve the rise of the color, such as on sensitized hair, and/or the condition of the fibers after coloration, for example, in the case of coloring with an oxidizing agent, and the resistance to shampooing of said colorations.

As used herein, the phrase “improvement in the condition of the fiber” means a reduction in the porosity or the alkaline solubility of the fiber and an improvement in at least one cosmetic property, for example, in the smoothness, softness and ease of disentangling and of styling.

This effect can be remanent, i.e., long-lasting.

The porosity is measured by fixing, at 37° C. and at pH 10, for two minutes, 2-nitro-para-phenylenediamine at 0.25% in an ethanol/pH 10 buffer mixture (10/90 volume ratio).

The alkaline solubility corresponds to the loss of mass of a sample of 100 mg of keratin fibers under the action of decinormal sodium hydroxide for 30 minutes at 65° C.

One new embodiment also relates to a coloring process comprising applying to human keratin fibers, such as hair, a composition comprising at least one aminosilicone comprising at least one aminoethylimino(C₄-C₈)alkyl group, in optionally rinsing the fibers, and then in applying at least one coloring composition chosen from oxidation dye compositions and direct dye compositions for a time that is sufficient to develop the color, optionally followed by a rinsing operation, then optionally by shampooing, and then drying.

Aminosilicones

The at least one aminosilicone comprising at least one aminoethylimino(C₄-C₈)alkyl group has, for example, the following formula:

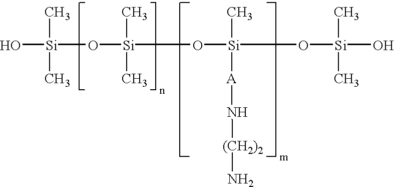

wherein:

A is chosen from linear and branched C₄-C₈ alkylene radicals, for example, C₄ alkylene radicals and

m and n are numbers such that the sum (n+m) can range, for example, from 1 to 2000 and, further for example, from 50 to 150, n can be a number ranging from 0 to 1999, for example, from 49 to 149, and m can be number ranging from 1 to 2000, for example, from 1 to 10.

The term “alkylene radical” means divalent saturated hydrocarbon-based groups.

The viscosity of the at least one aminosilicone, for example, can be greater than 25 000 mm²/s at 25° C.

For example, this viscosity can range from 30 000 to 200 000 mm²/s at 25° C. and further, for example, from 30 000 to 150 000 mm²/s at 25° C.

The viscosity of the at least one aminosilicone is measured at 25° C. according to the standard “ASTM 445 Appendix C.”

The at least one aminosilicone has a weight-average molecular mass, for example, ranging from 2000 to 1 000 000 and further, for example, from 3500 to 200 000.

The weight-average molecular masses of the at least one aminosilicone is measured by Gel Permeation Chromatography (GPC) at room temperature, as polystyrene equivalents. The columns used are styragel μ columns. The eluent is THF, and the flow rate is 1 ml/minute. 200 μl of a solution at 0.5% by weight of silicone in THF are injected. The detection is performed by refractometry and UV-metry.

One new embodiment involves using the at least one aminosilicone in the form of an oil-in-water emulsion. The oil-in-water emulsion may comprise at least one surfactant. The at least one surfactant may be of any nature, for example, cationic and/or nonionic.

The silicone particles in the emulsion may have a mean size ranging, for example, from 3 to 500 nanometers, and further, for example, from 5 to 300 nanometers, even further, for example, from 10 to 275 nanometers and even further, for example, from 150 to 275 nanometers. Such particle sizes are measured with a laser granulometer.

An example of a silicone corresponding to this formulation is DC2-8299® from the company Dow Corning.

Another new embodiment uses at least one aminosilicone in the pre-treatment composition in an amount ranging from 0.01% to 20% by weight relative to the total weight of the composition. For example, this amount may range from 0.1% to 15% by weight and further, for example, from 0.5% to 10% by weight relative to the total weight of the composition.

The pre-treatment composition may comprise any ingredient conventionally used in cosmetics, such as in the field of haircare. For example, it may comprise at least one additional surfactant and/or polymer. These surfactants and polymers may be chosen from nonionic, cationic, anionic and amphoteric surfactants and polymers. Among the additional polymers, aminosilicones other than those disclosed herein may, for example, be used.

The pre-treatment composition may have a pH ranging from 2 to 11 and, for example, from 4 to 9.

The pre-treatment composition may be in various forms, such as lotions, gels, creams, shampoos, sticks, mousses and sprays. For some of these forms, it may be packaged in a pump-dispenser bottle or in an aerosol container. In the case of an aerosol, the composition may be combined with a propellant that may be, for example, an alkane, or a mixture of alkane, dimethyl ether, nitrogen, nitrous oxide, carbon dioxide and haloalkanes, and also mixtures thereof.

In one new embodiment, the pre-treatment composition may be in shampoo form.

When the pre-treatment composition is in shampoo form, the composition comprises at least one surfactant, for example an anionic surfactant. The pre-treatment composition may also comprise a mixture of surfactants comprising at least one anionic surfactant and at least one other surfactant being chosen from nonionic and amphoteric surfactants.

The pre-treatment composition may be used in rinse-out or leave-in mode, i.e. its application may or may not be followed by a rinsing operation.

In one new embodiment, the acting time of the pre-treatment composition ranges from a few seconds to 60 minutes, for example, from 30 seconds to 15 minutes.

The application temperature of the pre-treatment composition may range from 10° C. to 70° C. For example, the application temperature may range from 10 to 60° C. such as at room temperature.

The nature and concentration of the dyes present in the dye compositions is not critical. In the case of colorations with direct dyes (in the presence or absence of oxidizing agents), the dye compositions may comprise at least one dye chosen from neutral, acidic and cationic nitrobenzene direct dyes, neutral, acidic and cationic azo and methine direct dyes, neutral, acidic and cationic quinone and, for example, anthraquinone direct dyes, azine direct dyes, triarylmethane direct dyes, indoamine direct dyes and natural direct dyes, and mixtures thereof.

In the case of colorations with oxidation dyes, the dye compositions may comprise at least one oxidation base.

The at least one oxidation base may be chosen from those conventionally used in oxidation dyeing, and among which mention may be made, for example, of orthophenylenediamines, para-phenylenediamines, double bases, orthoaminophenols, para-aminophenols, heterocyclic bases, and also their acid addition salts.

The oxidation dye compositions may also comprise at least one coupler.

Representatives of the at least one coupler can include, for example, meta-phenylenediamines, meta-aminophenols and meta-diphenols, mono- and polyhydroxylated naphthalene derivatives, sesamol and its derivatives, and heterocyclic compounds such as, for example, indole couplers, indoline couplers and pyridine couplers, and their acid addition salts.

The nature of the oxidizing agent used in the lightening direct dyeing (direct dyeing with an oxidizing agent) or in the oxidation dyeing is not critical. The at least one oxidizing agent may be chosen, for example, from hydrogen peroxide, urea peroxide, alkali metal bromates, alkali metal ferricyanides, and persalts, such as perborates and persulfates. At least one redox enzyme such as laccases, peroxidases and 2-electron oxidoreductases (such as uricase) may also be used as an oxidizing agent, where appropriate in the presence of the respective donor or cofactor thereof.

The examples that follow are intended to illustrate in a non-limiting way embodiments disclosed herein.

### EXAMPLES

The two pre-treatment compositions below were prepared. (expressed as grams of Active Material)

| Composition A | |
|---|---|
| Hydroxypropyl corn distarch phosphate | 3 |
| Hydroxyethylcellulose | 0.6 |
| Oxyethylenated (40 EO) hydrogenated castor oil | 0.5 |
| Polydimethylsiloxane: | 3.5 |
| DC2-8299 ® from the company Dow Corning | |
| Fragrance | 0.3 |
| Preserving agents | 0.3 |
| Demineralized water qs | 100 |
| Composition B | |
| Sodium lauryl ether sulfate comprising 2.2 mol of ethylene oxide | 7 |
| Cocoylbetaine | 2.5 |
| Glycol distearate | 1.5 |
| Polydimethylsiloxane: | 1.8 |
| DC2-8299 ® from the company Dow Corning | |
| Hydroxyethylcellulose quaternized with 2,3- | 0.4 |
| epoxypropyltrimethylammonium | |
| chloride, sold under brand name Ucare Polymer | |
| JR-400 ® by Union Carbide | |
| Acrylic polymer as an emulsion sold under | 0.8 |
| brand name Aqua SF1 ® by Noveon | |
| Preserving agents | qs |
| pH agents qs | pH 5 |
| Demineralized water qs | 100 |

Compositions A and B were applied for 15 minutes to locks of natural hair comprising 90% white hairs and to locks of hair sensitized by a bleaching operation.

After rinsing and drying, a coloration was performed on these locks and also on a control lock without pre-treatment, for 30 minutes.

Results: It was found:

that the condition of the fibers that had undergone the pre-treatment and the coloration was better than that of fibers that had undergone only the coloration process, which is reflected by softer, smoother, more individualized hair, that is easier to disentangle and to style;

that the rise in the coloration was better on the locks that had undergone the pre-treatment, for example, in the case of locks of sensitized hair.

In addition, the resistance of the color with respect to shampooing was satisfactory.

What is claimed is:

1. A process for coloring human keratin fibers comprising applying to said fibers a pre-treatment composition comprising at least one aminosilicone comprising at least one aminoethylimino(C₄-C₈)alkyl group, and thereafter, applying to said fibers, a composition comprising at least one coloring agent chosen from direct dyes and oxidation dyes.

2. The process according to claim 1, wherein said human keratin fibers are hair.

3. The process according to claim 1, wherein the least one aminosilicone comprising at least one aminoethylimino(C₄-C₈)alkyl group has the formula below: 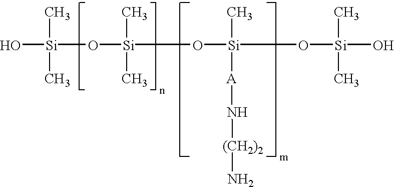
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000.

4. The process according to claim 3, wherein the sum (n+m) ranges from 50 to 150.

5. The process according to claim 3, wherein n is a number ranging from 49 to 149.

6. The process according to claim 3, wherein m is a number ranging from 1 to 10.

7. The process according to claim 3, wherein A is chosen from linear and branched C₄ alkylene radicals.

8. The process according to claim 1, wherein the viscosity of the at least one aminosilicone is greater than 25 000 mm²/s at 25° C.

9. The process according to claim 8, wherein the viscosity of the at least one aminosilicone ranges from 30 000 to 200 000 mm²/s at 25° C.

10. The process according to claim 9, wherein the viscosity of the at least one aminosilicone ranges from 30 000 to 150 0000 mm²/s at 25° C.

11. The process according to claim 1, wherein the at least one aminosilicone has a weight-average molecular mass ranging from 2000 to 1 000 000.

12. The process according to claim 11, wherein the at least one aminosilicone has a weight-average molecular mass ranging from 3500 to 200 000.

13. The process according to claim 1, wherein the at least one aminosilicone is in the form of an oil-in-water emulsion comprising at least one surfactant.

14. The process according to claim 13, wherein the oil-in water emulsion comprises at least one surfactant chosen from cationic and nonionic surfactants.

15. The process according to claim 13, wherein the particle size of the at least one aminosilicone ranges from 3 to 500 nanometers.

16. The process according to claim 15, wherein the particle size of said at least one aminosilicone ranges from 5 to 300 nanometers.

17. The process according to claim 16, wherein the particle size of said at least one aminosilicone ranges from 10 to 275 nanometers.

18. The process according to claim 17, wherein the particle size of said at least one aminosilicone ranges from 150 to 275 nanometers.

19. The process according to claim 1, wherein the at least one aminosilicone is present in an amount ranging from 0.01% to 20% by weight relative to the total weight of the composition.

20. The process according to claim 19, wherein the at least one aminosilicone is present in an amount ranging from 0.1% to 15% by weight relative to the total weight of the composition.

21. The process according to claim 20, wherein the at least one aminosilicone is present in an amount ranging from 0.5% to 10% by weight relative to the total weight of the composition.

22. The process according to claim 1, wherein the pre-treatment composition is provided in a form chosen from lotions, gels, creams, shampoos, sticks, mousses and sprays.

23. The process according to claim 1, wherein the pre-treatment composition is packaged in a pump-dispenser bottle or in an aerosol container.

24. The process according to claim 23, wherein the pre-composition is combined with at least one propellant chosen from alkanes, dimethyl ether, nitrogen, nitrous oxide, carbon dioxide and haloalkanes.

25. The process according to claim 1, wherein the pre-treatment composition comprises at least one surfactant chosen from nonionic, cationic, anionic and amphoteric surfactants.

26. The process according to claim 25, wherein the pre-treatment composition comprises a mixture of surfactants comprising at least one anionic surfactant and at least one other surfactant chosen from nonionic and amphoteric surfactants.

27. The process according to claim 1, wherein the pre-treatment composition further comprises at least one additional polymer other than said at least one aminosilicone comprising at least one aminoethylimino(C₄-C₈)alkyl group.

28. The process according to claim 27, wherein said at least one additional polymer is chosen from nonionic, cationic, anionic and amphoteric polymers.

29. The process according to claim 28, wherein said at least one additional polymer is an aminosilicone different than said at least one aminosilicone.

30. The process according to claim 1, wherein the pH of the pre-treatment composition ranges from 2 to 11.

31. The process according to claim 30, wherein the pH of the pre-treatment composition ranges from 4 to 9.

32. A process according to claim 3, wherein said human keratin fibers are hair.

33. A process according to claim 1, wherein the pre-treatment composition is left to act for a time ranging from a few seconds to 60 minutes.

34. A process according to claim 33, wherein the pre-treatment composition is left to act for a time ranging from 30 seconds to 15 minutes.

35. A process for improving the rise of coloration on human keratin fibers colored with at least one coloring agent chosen from oxidation dyes and direct dyes, comprising, prior to coloring said fibers with said at least one coloring agent, applying to said fibers a pre-treatment composition comprising at least one aminosilicone comprising at least one aminoethylimino(C₄-C₈)alkyl group.

36. A process according to claim 35, wherein said human keratin fibers are sensitized hair.

37. A process for improving the condition of human keratin fibers after coloration with at least one coloring agent chosen from oxidation dyes and direct dyes, comprising, prior to coloring said fibers with said at least one coloring agent chosen from oxidation dyes and direct dyes, applying to said fibers a pre-treatment composition comprising at least one aminosilicone comprising at least one aminoethylimino(C₄-C₈)alkyl group.

38. The process according to claim 37, wherein said at least one coloring agent comprises an oxidizing agent.

39. A process for reducing the porosity of human keratin fibers after coloration with at least one coloring agent chosen from oxidation dyes and direct dyes, comprising, prior to coloring said fibers with said at least one coloring agent, applying to said fibers a pre-treatment composition comprising at least one aminosilicone comprising at least one aminoethylimino(C₄-C₈)alkyl group.

40. A process for reducing the alkaline solubility of human keratin fibers after coloration with at least one coloring agent chosen from oxidation dyes and direct dyes, comprising, prior to coloring said fibers with said at least one coloring agent, applying to said fibers a pre-treatment composition comprising at least one aminosilicone comprising at least one aminoethylimino(C₄-C₈)alkyl group.

41. A process for improving the resistance to shampooing of coloration on human keratin fibers, comprising, prior to coloring said fibers with at least one coloring agent chosen from oxidation dyes and direct dyes, applying to said fibers a pre-treatment composition comprising at least one aminosilicone comprising at least one aminoethylimino(C₄-C₈)alkyl group.

42. A composition for pre-treatment of human keratin fibers before coloration with at least one coloring agent chosen from oxidation dyes and direct dyes, said composition comprising at least one aminosilicone comprising at least one aminoethylimino(C₄-C₈)alkyl group, wherein said composition is effective for the pre-treatment of human keratin fibers prior to a process for coloring with said at least one coloring agent.

43. A process for coloring human keratin fibers comprising
applying to said fibers a pre-treatment composition comprising at least one aminosilicone comprising at least one aminoethylimino(C₄-C₈)alkyl group,
optionally rinsing said fibers,
applying a composition comprising at least one coloring agent chosen from direct dyes and oxidation dyes to said fibers,
leaving said at least one coloring agent on said fibers for a time sufficient to develop the color, and
optionally rinsing and optionally drying said fibers.
